(19) United States
(12) Patent Application Publication
(10) Pub. No.: US 2012/0071975 A1
(43) Pub. Date: Mar. 22, 2012

(54) DEVICE AND METHOD FOR ASSISTING IN FLEXOR TENDON REPAIR AND REHABILITATION

(76) Inventor: Eduardo Gonzalez-Hernandez, Coconut Grove, FL (US)

(21) Appl. No.: 13/229,235

(22) Filed: Sep. 9, 2011

Related U.S. Application Data
(60) Provisional application No. 61/403,116, filed on Sep. 10, 2010.

(51) Int. Cl. A61F 2/08

(52) U.S. Cl. 623/13.11

(57) ABSTRACT

A device and method used to protect at least one end of a severed flexor tendon from contacting the edge of a flexor tendon pulley. The device, in one embodiment, can include a body forming a complete perimeter around at least a portion of a cavity passing through the device. The body of the device includes a first portion that is adapted for insertion through the passage of a flexor tendon pulley, a second portion that adapted funnel the severed flexor tendon into the cavity, and a perforation that extends the length of the body that is adapted to be torn along the length thereof to afford removal of the device from around the previously-severed flexor tendon.

This application claims the benefit of U.S. Provisional Application No. 61/403,116, filed Sep. 10, 2010, which is incorporated herein by reference.

### BACKGROUND OF THE INVENTION

1. Field of the Invention

The present invention is generally related to a device and method for use thereof for facilitating repair of a severed flexor tendon. More particularly, the present invention is related to a device and method for use thereof for facilitating movement of a severed flexor tendon end and/or a repaired flexor tendon with respect to a flexor tendon pulley. More specifically, the present invention relates to a device and method thereof for protecting both a severed flexor tendon end and a repaired flexor tendon from inflammation and/or damage from contact with edges of a flexor tendon pulley.

2. Description of the Prior Art

Fingers in the hand are moved by flexor and extensor tendons arising from muscles in the forearm. The mechanism facilitating movement of the fingers using the flexor tendons includes three main components in each of the fingers: 1) the skeleton, including phalanges and corresponding articulations (or interphalangeal joints) between the phalanges; 2) a flexor tendon sheath; and 3) the flexor tendons themselves.

In each finger, two flexor tendons work to flex the proximal and distal interphalangeal joints, namely the flexor digitorum superficialis and the flexor digitorum profundis, respectively. Furthermore, the flexor tendon sheath holds the flexor tendons in close proximity to the phalanges of each finger to ensure that their pull produces immediate movement at the interphalangeal joints. In the absence of the flexor tendon sheaths, the flexor tendons simply “bow-string” and fail to produce the desired joint motion.

The flexor tendon sheath is highly specialized tissue that is anchored to bone and forms a very smooth but tight fibrous tunnel around the flexor tendons. With the flexor tendons surrounded by the flexor tendon sheath, there is a measurable hydrostatic pressure within the substance of the tendons. The flexor tendon sheath is not a uniform fibrous tunnel, but rather, is made of identifiable individual segments. The individual segments that make up the flexor tendon sheath are referred to as pulleys because of the mechanical role they play: i.e., holding the flexor tendons close to the phalanges; preventing “bow-stringing” of the flexor tendons; and ultimately translating pull of the flexor tendons into joint motion.

For decades, surgical repair of severed flexor tendons has proven challenging. When the flexor tendons of a finger are severed, the flexor tendons retract through the flexor tendon sheath. Thus, to facilitate repair thereof, a severed flexor tendon end is passed through at least one flexor tendon pulley to a repair site facilitating repair thereof. Furthermore, after repair of the flexor tendon, a joint on the repaired flexor tendon formed at the repair site must be passed through at least one flexor tendon pulley. However, passage of the severed flexor end and the joint through the flexor tendon pulley forces contact with one or more edges thereof. Such contact can cause inflammation and/or damage to the severed flexor end and the joint, and can limit the potential for the severed flexor tendon to heal.

Therefore, there is a need for a device and method for use thereof for facilitating movement of a severed flexor tendon end and/or a repaired flexor tendon with respect to a flexor tendon pulley. Such a device and method for use thereof should provide protection for both a severed flexor tendon end and a repaired flexor tendon from inflammation and/or damage from contact with edges of a flexor tendon pulley.

### SUMMARY OF THE INVENTION

The present invention in one preferred embodiment contemplates a device for protecting at least one end of a severed flexor tendon from contacting an edge a flexor tendon pulley, the device including a body formed from a material suitable for implantation, the body having a proximal end, an opposite distal end, and a length therebetween, the body having a cavity defined by a first portion and a second portion, the first portion extending from the distal end to the second portion and being configured for insertion through a passage of the flexor tendon pulley, the second portion extending from the first portion to the proximal end and being at least in part arcuate and adapted for placement adjacent the edge of the flexor tendon pulley, at least a portion of the body configured to encircle at least a portion of the severed flexor tendon inserted therethrough, the body being perforated along the length thereof.

The present invention in a further preferred embodiment contemplates an implant for implantation into a human hand for protecting at least one end of a severed flexor tendon from contacting an edge of a flexor tendon pulley, the implant including a lip portion formed from a material suitable for implantation into the human hand, the lip portion being at least in part arcuate and adapted for placement in contact with the edge of the flexor tendon pulley, the lip portion having a channel defined by a first portion, a second portion, and a connecting portion extending between the first portion and the second portion, the channel configured to receive the edge of the flexor tendon pulley, where, when the edge is received in the channel, the first portion contacts the interior of the flexor tendon pulley and the second portion contacts the exterior of the flexor tendon pulley.

The present invention in a further preferred embodiment contemplates a device for protecting at least one end of a severed flexor tendon from contacting an edge of a flexor tendon pulley, the implant including a cavity extending through the device, the cavity adapted to receive the severed flexor tendon therethrough; a body forming a complete perimeter around at least a portion of the cavity, the body having a first portion, a second portion, and a mid-longitudinal axis extending through the first portion and the second portion, the first portion having a cross-section transverse to the mid-longitudinal axis, the second portion having a cross-section transverse to the mid-longitudinal axis, the first portion adapted to be inserted into the flexor tendon pulley, and a second portion adapted to funnel the severed flexor tendon into the cavity, the cross-section of the second portion being greater than the cross-section of the first portion; and a perforation formed through the body, the perforation having a length extending along the first and second portions, the perforation adapted to be torn along the length thereof, where, when the perforation is torn, the complete perimeter of the body is eliminated to afford removal of the device from around a previously-severed flexor tendon.

The present invention in a further preferred embodiment contemplates A method for reattaching severed ends of a flexor tendon, the method including providing a device having a cavity, a body forming a complete perimeter around at least a portion of the cavity, and a perforation formed through the body, the perforation having a length extending along the body; inserting at least a portion of the device through a flexor tendon pulley; inserting one of the severed ends of the flexor tendon through the cavity; reattaching the severed ends of the flexor tendon; tearing the perforation along the length thereof to eliminate the complete perimeter around the at least a portion of the cavity; and removing the device from around the reattached flexor tendon.

It is understood that both the foregoing general description and the following detailed description are exemplary and exemplary only, and are not restrictive of the invention as claimed.

### BRIEF DESCRIPTION OF THE DRAWINGS

The accompanying drawings, which are incorporated in and constitute a part of the specification, illustrate preferred embodiments of the invention. Together with the description, they serve to explain the objects, advantages and principles of the invention. In the drawings:

FIG. 1 is a side perspective view of a sutured tendon end being forced into a passage through a flexor tendon pulley;

FIG. 2 is a side perspective view of the sutured tendon end depicted in FIG. 1 with corresponding damage after an unsuccessful attempt at forcing the tendon end into the flexor tendon pulley;

FIG. 3 is a side perspective view of a bulky repair site along a reattached tendon positioned between two flexor tendon pulleys;

FIG. 4 is a side perspective view of the bulky repair site along the reattached tendon abutting the edge of one of the two flexor pulleys depicted in FIG. 3;

FIG. 5 is a partially rearward side perspective view of a first device according to the present invention;

FIG. 6 is a top plan view of the first device depicted in FIG. 5;

FIG. 7 is a partially rearward side perspective view of a second device according to the present invention;

FIG. 8 is a top plan view of the second device depicted in FIG. 7;

FIG. 9 is a partially rearward side perspective view of a third device according to the present invention;

FIG. 10 is a partially rearward side perspective view of a fourth device according to the present invention;

FIG. 11 is a partially rearward side perspective view of the fourth device implanted with respect to a flexor tendon pulley with portions of the fourth device shown in phantom;

FIG. 12 is a partially rearward side perspective view of a fifth device according to the present invention;

FIG. 13 is a partially rearward side perspective view of the fifth device implanted with respect to a flexor tendon pulley with portions of the fifth device shown in phantom;

FIG. 14 is a perspective view of a sixth device with a perforation along the length thereof implanted with respect to a flexor tendon pulley with the severed ends of a flexor tendon on either side of the sixth device;

FIG. 15 is a perspective view of the sixth device implanted with respect to the flexor tendon pulley with sutures extending through the sixth device from one of the severed ends to adjacent the other of the severed ends;

FIG. 16 is a perspective view of the sixth device implanted with respect to the flexor tendon pulley with the severed ends reattached to one another;

FIG. 17 is a perspective view of the sixth device implanted with respect to the flexor tendon pulley with the perforation along the length thereof initially torn;

FIG. 18 is a perspective view of the sixth device implanted with respect to the flexor tendon pulley with the perforation along the length thereof torn a further amount from FIG. 17;

FIG. 19 is a perspective view of the sixth device implanted with respect to the flexor tendon pulley with the perforation along the length thereof torn a still further amount from FIG. 18; and

FIG. 20 is a perspective view of the sixth device removed from the flexor tendon pulley with the perforation torn completely along the length thereof.

### DETAILED DESCRIPTION OF THE PREFERRED EMBODIMENT

The following description is intended to be representative only and not limiting, and many variations can be anticipated according to these teachings. Reference will now be made in detail to the preferred embodiments of this invention, examples of which are illustrated in the accompanying drawings.

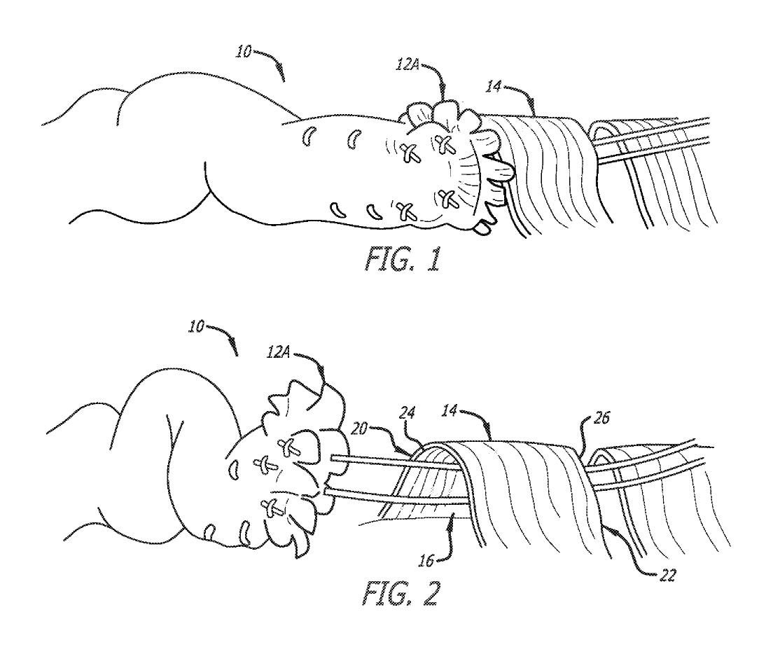

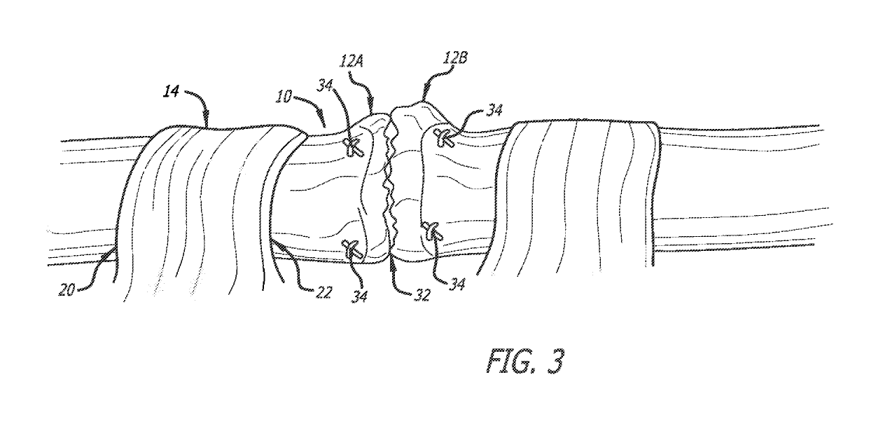

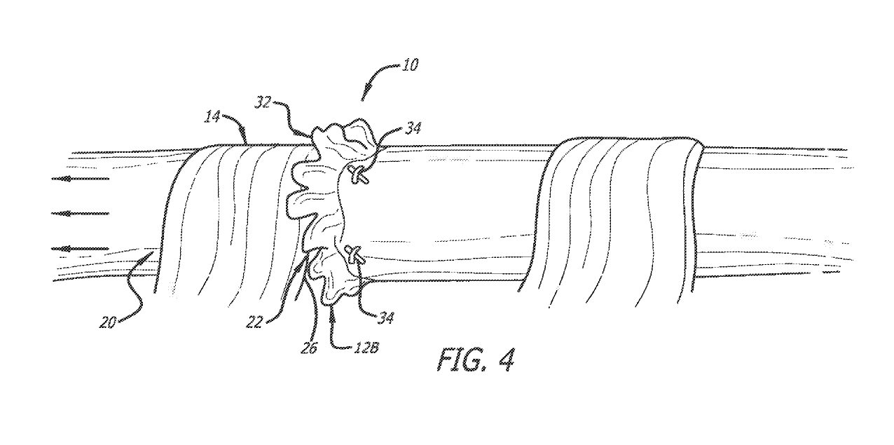

A severed tendon 10 includes severed tendon ends 12A (FIGS. 1-3) and 12B (FIGS. 3 and 4). Because of the damage thereto, severed tendon ends 12A and 128 are inflamed. The inflammation of severed tendon ends 12A and 128 inhibits passage thereof through a flexor tendon pulley 14, and makes rejoining severed tendon ends 12A and 12B problematic, flexor tendon pulley 14 includes a passage 16 for receiving severed tendon 10 therethrough. Passage 16 includes a first opening 20 and a second opening 22, and corresponding edges 24 and 26 of flexor tendon pulley 14 surround first and second openings 20 and 22.

Due to the inflammation thereof, severed tendon ends 12A and 128 can be larger than first opening 20. The enlarged size (relative to the remainder of severed tendon 10) of severed tendon end 12A, for example, inhibits passage thereof through passage 16. Thus, attempting to pass severed tendon ends 12A and 12B into first and second openings 20 and 22, respectively, will force severed tendon ends 12A and 12B against corresponding edges 24 and 26. As depicted in FIG. 1, the contact of severed tendon end 12A with edge 24 can cause abrasion and damage thereto. Attempts to pass severed tendon end 12A into first opening 20 or severed tendon end 128 into second opening 22 can result in further inflammation, and makes rejoining severed tendon ends 12A and 12B even more problematic.

The inflammation of severed tendon ends 12A and 128 can make a joint 32 (FIG. 3) formed by sutured connection therebetween difficult to pass through passage 16. As depicted in FIG. 3, joint 32 between severed tendon ends 12A and 12B formed with the use of sutures 34 is enlarged with respect to the remainder of the previously-severed tendon 10. For example, as depicted in FIG. 4, joint 32 can be larger than second opening 22 of flexor tendon pulley 14. Thus, attempting to pass joint 32 into second opening 22 will force joint 32 against edge 26. Contact of joint 32 with edge 26 can cause abrasion and damage thereto resulting in further inflammation. Given that normal movement of previously-severed tendon 10 will force joint 32 into and out of passage 16, abrasion and damage thereto can continue after the surgery is complete, thereby hindering recovery.

The embodiments of an implantable device described below serve in aiding passage of severed tendon ends 12A and 12B and/or joint 32 through passage 16. In doing so, the embodiments of the implantable device serve in funneling severed tendon ends 12A and 12B and/or joint 32 into passage 16. As discussed below, the implantable devices serve in covering at least one of corresponding edges 24 and 26 of first and second openings 20 and 22, respectively. As such, the embodiments of the implantable device facilitate gliding of flexor tendon 10 in either the severed or the previously-severed state through one of first and second openings 20 and 22 of passage 16.

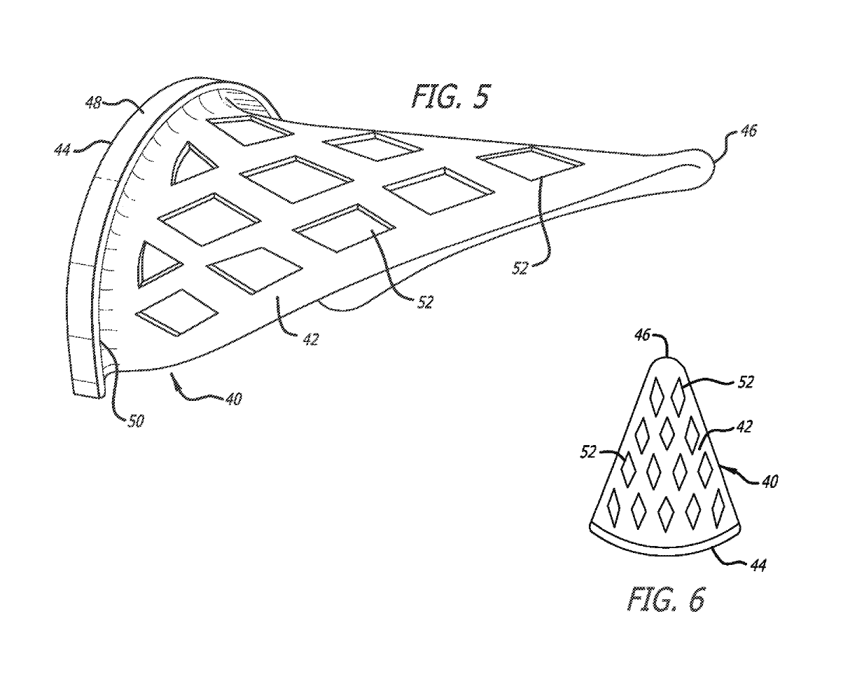

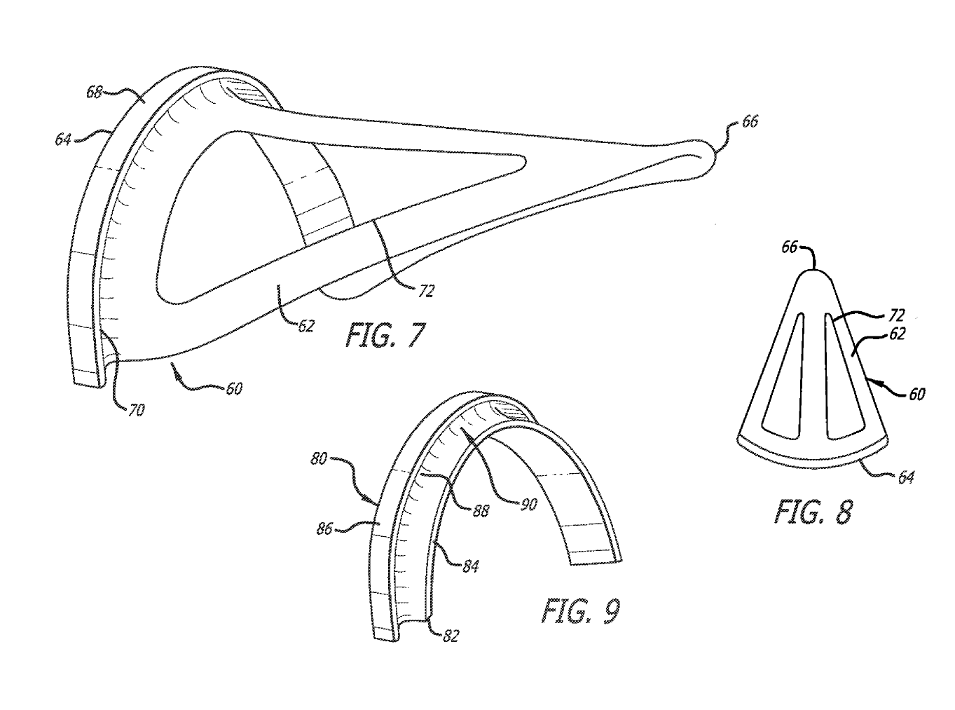

An implantable device 40 is depicted in FIGS. 5 and 6 and an implantable device 60 is depicted in FIGS. 7 and 8. Both of devices 40 and 60 can be implanted with respect to flexor tendon pulley 14. In part, the shapes of devices 40 and 60 conform to the interior shape of passage 16 through flexor tendon pulley 14. To that end, devices 40 and 60 include body portions 42 and 62, respectively, shaped as a partial funnel or cone. The partial funnel or cone shape of body portions 42 and 62 can be formed as approximately half of a complete cone. Thus, the shape of body portions 42 and 62 is akin to splitting a funnel or cone along a plane parallel or slightly transverse to the longitudinal axis thereof. Alternatively, the shape of body portions 42 and 62 may take the form of a truncated cylinder.

Body portions 42 and 62 include first ends 44 and 64 and second ends 46 and 66, respectively. First ends 44 and 64 include the maximum dimensions of respective body portions 42 and 62, and second ends 46 and 66 include the minimum dimensions of respective body portions 42 and 62. Second ends 46 and 66 correspond to apexes of the partial funnel or cone shapes of body portions 42 and 62, respectively. Thus, the preferably rounded second ends 46 and 66 facilitate entry of body portions 42 and 62 into passage 16 of flexor tendon pulley 14.

Furthermore, lips 48 and 68 are provided at first ends 44 and 64 of body portions 42 and 62, respectively. Lips 48 and 68 can include portions extending outwardly from body portions 42 and 62, and portions extending toward second ends 46 and 64, respectively. With portions of body portions 42 and 62, lips 48 and 68 form respective cavities 50 and 70. Cavities 50 and 70 have at least in part U-shaped cross sections in planes perpendicular to body portions 42 and 62 running from first ends 44 and 64 to second ends 46 and 66. As discussed below, cavities 50 and 70 are used to receive one of edges 24 and 26.

Devices 40 and 60 can be made of either non-absorbable or absorbable materials. For example, the non-absorbable materials can include nylon, and the absorbable materials can include polylactic acid or polygylcolic add. When made of absorbable materials, devices 40 and 60 can include fenestrations (or openings) 62 and 72, respectively, to aid the absorption thereof. Fenestrations 52 and 72 decrease the amount of material required to be absorbed, and thus, decrease the time required to complete absorption. Furthermore, devices 40 and 60 can be impregnated with a lubricant such as hyaluronic acid to aid movement (and thereby limit abrasion or damage) of severed tendon ends 12A and 12B and/or joint 32 that comes into contact with devices 40 and 60.

During use, each of first and second openings 20 and 22 can receive one of devices 40 and 60. In positioning devices 40 and 60, second ends 46 and 66 thereof are inserted into least one of first and second openings 20 and 22 of passage 16. Second ends 46 and 66 are inserted into passage 16 to depths affording the positioning of first ends 44 and 64 adjacent edge s 24 or 26. In doing so, edges 24 and 26 are cradled in cavities 50 and 70 formed by the at least in part U-shaped cross sections of lips 48 and 68, respectively.

Lips 48 and 68 protect severed tendon ends 12A and 12B and/or joint 32 from contact with edges 24 and 26. Thus, when passed through passage 16, severed tendon ends 12A and 12B and/or joint 32 contacts devices 40 and 60 rather than edges 24 and 26. In doing so, devices 40 and 60 allow severed tendon ends 12A and 12B and/or joint 32 to glide through passage 16. Gliding of severed tendon ends 12A and 126 and/or joint 32 past edges 24 and 26 serves to minimize the above-discussed abrasion or damage. If material forming devices 40 and 60 is impregnated therewith, the lubricant serves in aiding the gliding movement of severed tendon ends 12A and 12B and/or joint 32 with respect to devices 40 and 60.

An implantable device 80 is depicted in FIG. 9, and is formed from a channel 82 shaped into an arc. The arcuate shape of device 80 can be formed, for example, as a half circle or a half ellipse. In cross section, channel 82 forming device 80 is at least in part U-shaped, and includes a first portion 84, a second portion 86, and a connecting portion 88.

First portion 84 and second portion 86 can have identical or different lengths along the cross section of device 80. As depicted in FIG. 9, for example, first portion 84 is longer than second portion 86. When first portion 84 and second portion 86 have different lengths, channel 80 would have a J-shape in cross section. However, the lengths of first portion 84 and second portion 86 would have lengths that in a preferred embodiment would not exceed the length of a human pulley along the cross section of device 80. The lengths of first and second portion 84 and 86 will typically not exceed 2 mm.

First portion 84, second portion 86, and connecting portion 88 form a cavity 90 for receiving edges 24 and 26 of first and second openings 20 and 22, respectively. Each of first and second openings 20 and 22 can receive one of devices 80. Given that channel 82 is arcuate, first portion 84 is provided at the interior side of the arc and second portion 86 is provided at the exterior side of the arc. Thus, when funneling device 80 is implanted, first portion 84 abuts the interior of flexor tendon pulley 14, and second portion 86 abuts exterior of flexor tendon pulley 14. To prevent dislodgement, devices 80 can be sutured in position relative to first and second openings 20 and 22.

If one of devices 80, for example, is received in first opening 20, edge 24 would be received in cavity 90 to protect severed tendon ends 12A and 12B and/or joint 32. In doing so, first portion 84 would be positioned to contact the inner surface of flexor tendon pulley 14, and second portion 86 would be positioned to contact the outer surface of flexor tendon pulley 14. Besides protecting severed tendon ends 12A and 12B and/or joint 32 from contact with edge 24, device 80 would support flexor tendon pulley 14 at opening 20.

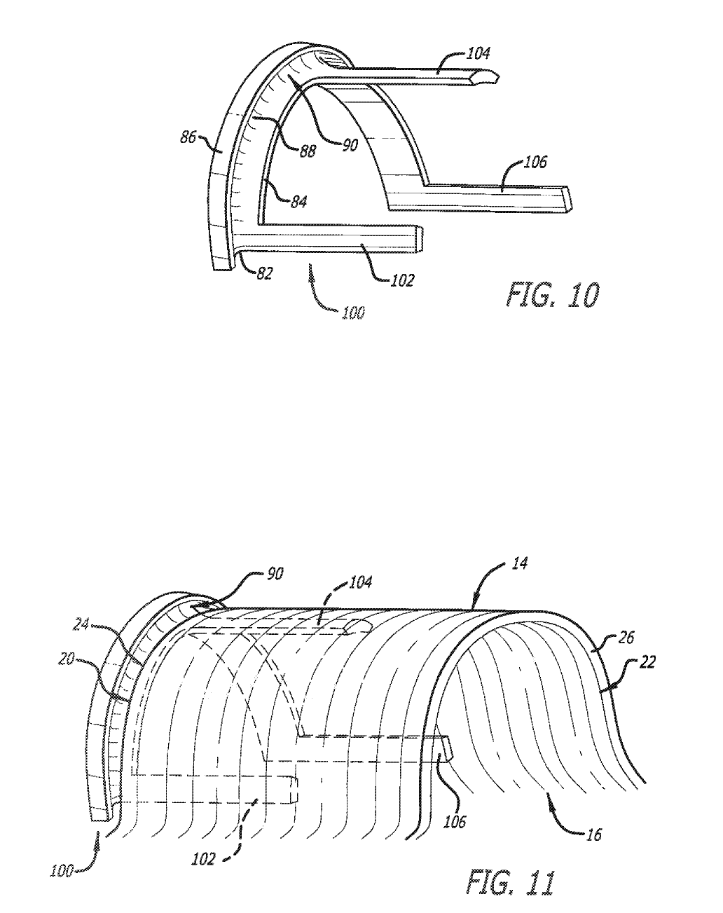

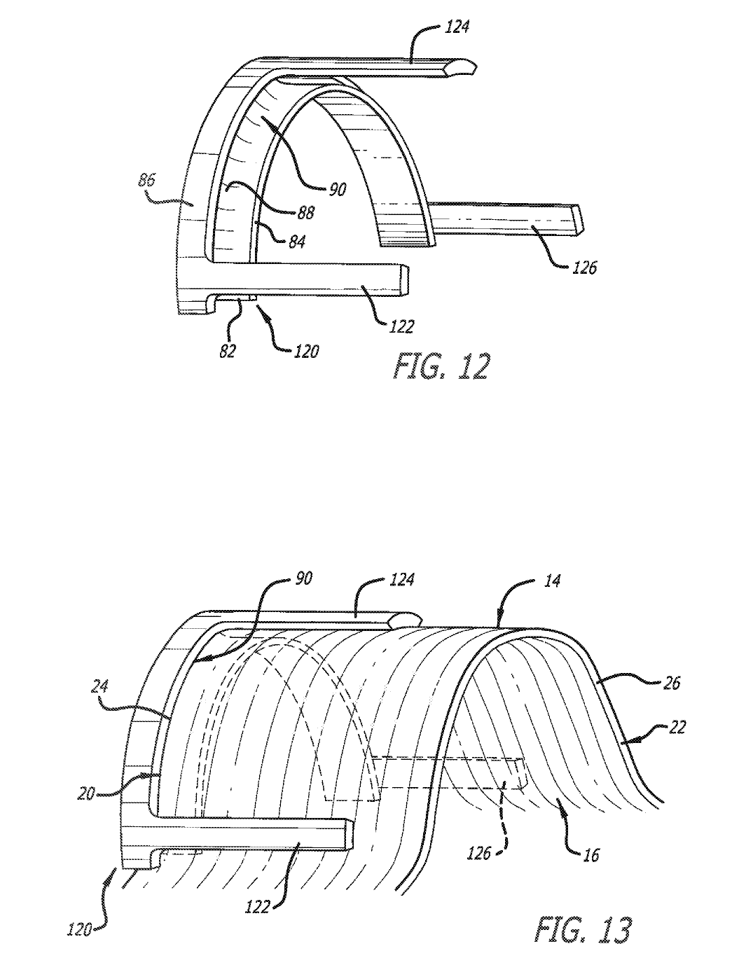

To provide additional support to flexor tendon pulley 14, implantable devices 100 (FIGS. 10 and 11) and 120 (FIGS. 12 and 13) are provided, devices 100 and 120 are each formed in part by channel 82 of device 80. Thus, designations identical to those used in describing channel 82 of device 80 will be use to describe channel 82 of devices 100 and 120. Furthermore, devices 40 and 60 can be modified to be formed in part by channel 82. Rather than having lips 48 and 68, devices 40 and 60 can instead include channel 82.

As discussed below, devices 100 and 120 each include legs extending outwardly from channel 82. Legs of devices 100 and 120 can, for example, extend parallelly with respect to one another, and are used for stabilizing channel 82 relative to flexor tendon pulley 14, thereby limiting the need to suture devices 100 and 120 in position. Similar to device 80, each of first and second openings 20 and 22 can receive one of devices 100 and 120.

In addition to channel 82, device 100 includes legs 102, 104, and 106. Legs 102, 104, and 106 are provided to stabilize channel 82, and extend outwardly from first portion 84. As discussed above, first portion 84 is provided to contact the inner portion of edges 24 and 26. Thus, legs 102, 104, and 106 are provided to be inserted into passage 16 to abut the inner surface thereof, in doing so, legs 102, 104, and 106 serve to support flexor tendon pulley 14.

In addition to channel 82, device 120 includes legs 122, 124, and 126. Legs 122, 124, and 126 are provided to stabilize channel 82, and extend outwardly from second portion 86. As discussed above, second portion 86 is provided to contact the outer portion of edges 24 and 26. Thus, legs 122, 124, and 126 are not provided to be inserted into passage 16. Instead, legs 122, 124, and 126 are provided to abut the outer surface of flexor tendon pulley 14. As such, legs 122, 124, and 126 do not contact severed tendon ends 12A and 12B and/or joint 32 which are pulled through flexor tendon pulley 14.

In further embodiments, devices 100 and 120 can be modified to include legs 102, 104, and 106 and legs 122, 124, and 126. In such embodiments, legs 102, 104, and 106, and legs 122, 124, and 136, respectively, would be opposed to one another, and be configured to receive flexor tend pulley 14 therebetween.

Similar to devices 40 and 60, devices 80, 100, and 120 can be made of either non-absorbable or absorbable materials. Again, the non-absorbable materials can include nylon, and the absorbable materials can include polylactic acid or polyglycolic acid. Furthermore, devices 80, 100, and 120 can be impregnated with a lubricant such as hyaluronic acid to aid movement (and thereby limit abrasion or damage) of severed tendon ends 12A and 12B and/or joint 32 that comes into contact therewith.

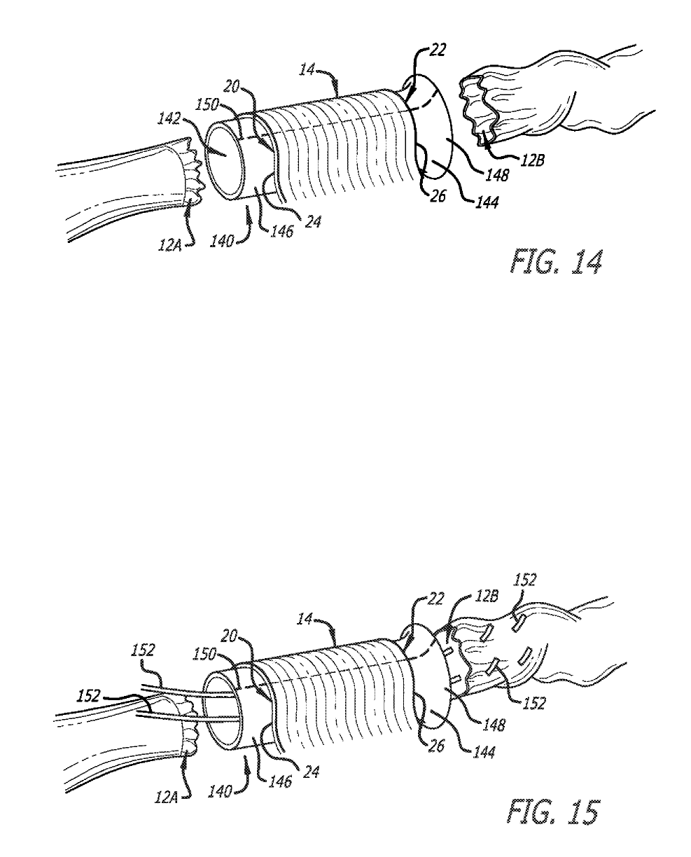

A device 140 includes a complete perimeter surrounding at least a portion of a cavity 142, and is sized to partially extend through passage 16 of flexor tendon pulley 14, device 140 includes body 144 through which cavity 142 is formed. As depicted in FIG. 14, the complete perimeter extends along the length of body 144. However, body 144 may include one or more fenestrations (not shown) formed therethrough. Thus, if body 144 includes one or more fenestrations, the complete perimeter surrounding cavity 142 may be interrupted.

Body 144 includes a first portion 146 and a second portion 148, and cavity 142 is formed therethrough. First portion 146 is sized to fit into cavity 16 flexor tendon pulley 14, and second portion 148 is larger than first portion 146. Thus, second portion 148 remains outside of cavity 16 of flexor tendon pulley 14. As such, cavity 142 is larger through second portion 148, than through first portion 146. For example, as depicted in FIG. 14, first portion 146 has a cylindrical shape, and second portion 148 has a frusto-conical shape. Accordingly, severed flexor tendon 12B, for example, can initially be fed into cavity 142 at second portion 148, and then funneled into the remainder of cavity 142 through first portion 146. Thereafter, severed tendon end 12B can be pulled through cavity 142 and ultimately reattached to severed tendon end 12A.

Once severed tendon ends 12A and 12B are reattached, device 140 can be removed using a perforation 150. Perforation 150 is formed through body 144, and has a length extending along first portion 146 and second portion 148. When torn along perforation 150, the complete perimeter around cavity 142 is eliminated. Without having a complete perimeter around cavity 142, device 140 can be removed from around previously-severed tendon 10.

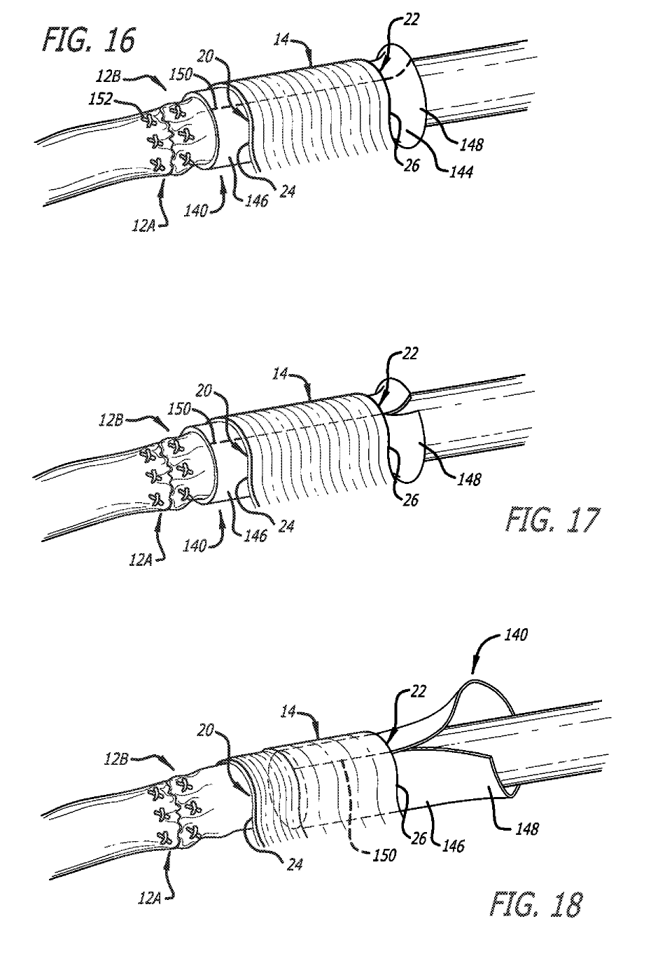

During use to afford reattachment of severed tendon ends 12A and 128, device 140 is inserted into passage 16 of flexor tendon pulley 14. As depicted in FIG. 14, device 140 is inserted into second opening 22 of passage 16. In doing so, first portion 146 is inserted into passage 16, and second portion 148 is positioned adjacent second opening 22. As depicted in FIG. 15, sutures 152 are then inserted through cavity 142 of device 140, and attached to severed tendon end 12B. Sutures 152 allow severed tendon end 128 to be pulled through cavity 142 (and hence, passage 16) to be positioned adjacent first opening 20. Thereafter, as depicted in FIG. 16, severed tendon ends 12A and 128 can be reattached to one another.

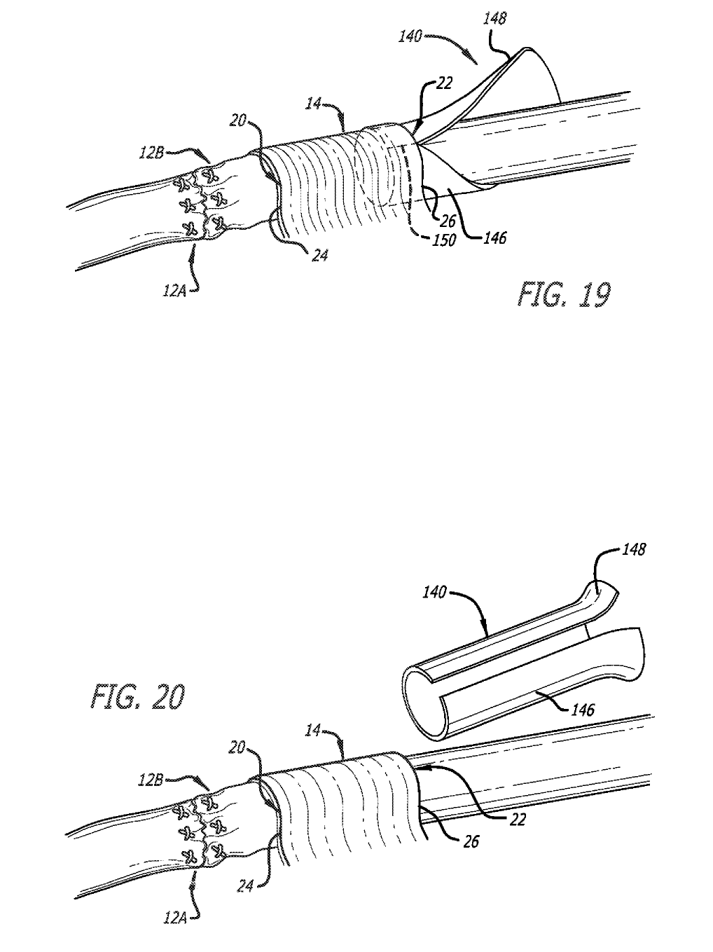

To facilitate removal thereof from passage 16, a small portion of device 140, as depicted in FIG. 17, is initially removed from passage 16 and perforation 150 is torn along the removed length of device 140. As depicted in FIGS. 18 and 19, progressively larger portions of device 140 are removed from passage 16 and perforation 10 is torn along the progressively longer removed lengths of device 140. Once perforation 150 is completely torn along the length thereof, device 140 can be removed from passage 16 and from around previously-severed tendon 10.

Similar to devices 40, 60, 80, 100, and 120, device 140 can be made of either non-absorbable or absorbable materials. Again, the non-absorbable materials can include nylon, and the absorbable materials can include polylactic acid or polyglycolic acid. Furthermore, device 140 can be impregnated with a lubricant such as hyaluronic acid to aid movement (and thereby limit abrasion or damage) of severed tendon ends 12A and 128 and/or joint 32 that comes into contact therewith.

Other embodiments of the invention will be apparent to those skilled in the art from consideration of the specification and practice of the invention disclosed herein. Accordingly, it is intended that the specification and examples be considered as exemplary only, with a true scope and spirit of the invention being indicated by the following claims.

We claim:

1. A device for protecting at least one end of a severed flexor tendon from contacting an edge of a flexor tendon pulley, the device comprising:
a body formed from a material suitable for implantation, said body having a proximal end, an opposite distal end, and a length therebetween, said body having a cavity defined by a first portion and a second portion, said first portion extending from said distal end to said second portion and being configured for insertion through a passage of the flexor tendon pulley, said second portion extending from said first portion to said proximal end and being at least in part arcuate and adapted for placement adjacent the edge of the flexor tendon pulley, at least a portion of said body configured to encircle at least a portion of the severed flexor tendon inserted therethrough, said body being perforated along the length thereof.

2. The device according to claim 1, wherein said second portion is configured to extend beyond a second edge of the flexor tendon pulley opposite the edge adjacent said first portion support the interior of the flexor tendon pulley to prevent collapse thereof.

3. The device according to claim 1, wherein at least a portion of the body is impregnated with a lubricant.

4. The device according to claim 1, wherein said second portion is substantially cylindrical.

5. The device according to claim 1, wherein said body is bioresorbable.

6. The device according to claim 5, wherein said body has fenestrations.

7. A device for protecting at least one end of a severed flexor tendon from contacting an edge of a flexor tendon pulley, the device comprising:
a cavity extending through the device, said cavity adapted to receive the severed flexor tendon therethrough;
a body forming a complete perimeter around at least a portion of said cavity, said body having a first portion, a second portion, and a mid-longitudinal axis extending through said first portion and said second portion, said first portion having a cross-section transverse to the mid-longitudinal axis, said second portion having a cross-section transverse to the mid-longitudinal axis, said first portion adapted to be inserted into the flexor tendon pulley, and said second portion adapted to funnel the severed flexor tendon into the cavity, said cross-section of said second portion being greater than said cross-section of said first portion; and
a perforation formed through said body, said perforation having a length extending along said first and second portions, said perforation adapted to be torn along the length thereof, wherein, when said perforation is torn, said complete perimeter of said body is eliminated to afford removal of the device from around a previously-severed flexor tendon.

8. The device according to claim 7, wherein said second portion has a frusto-conical shape.

9. The device according to claim 7, wherein said second portion includes a first end and a second end, and said second portion tapers from said first end toward said second end thereof.

10. The device according to claim 9, wherein said second end of said second portion abuts said first portion.

11. The device according to claim 7, wherein said cavity at said second portion has a larger dimension transverse to the mid-longitudinal axis than said cavity at said first portion.

12. A method for reattaching severed ends of a flexor tendon, the method comprising:
providing a device having a cavity, a body forming a complete perimeter around at least a portion of the cavity, and a perforation formed through the body, the perforation having a length extending along the body;
inserting at least a portion of the device through a flexor tendon pulley;
inserting one of the severed ends of the flexor tendon through the cavity;
reattaching the severed ends of the flexor tendon;
tearing the perforation along the length thereof to eliminate the complete perimeter around the at least a portion of the cavity; and
removing the device from around the reattached flexor tendon.

13. The method according to claim 12, wherein inserting one of the severed ends of the flexor tendon through the cavity further comprises funneling the one of the severed ends into the cavity.

14. The method according to claim 12, wherein the device includes a first portion and a second portion, the first portion being adapted to be inserted into the flexor tendon pulley, and the second portion being adapted to funnel the one of the severed ends into the cavity.

15. An implant for implantation into a human hand for protecting at least one end of a severed flexor tendon from contacting an edge of a flexor tendon pulley, the implant comprising:
a lip portion formed from a material suitable for implantation into the human hand, said lip portion being at least in part arcuate and adapted for placement in contact with the edge of the flexor tendon pulley, said lip portion having a channel defined by a first portion, a second portion, and a connecting portion extending between said first portion and said second portion, said channel configured to receive the edge of the flexor tendon pulley, wherein, when the edge is received in said channel, said first portion contacts the interior of the flexor tendon pulley and said second portion contacts the exterior of the flexor tendon pulley; and
at least one leg extending outwardly from one of said first portion and said second portion, said at least one leg portion aligned with said first portion and adapted to contact the interior of said flexor tendon pulley when extending outwardly from said first portion, and aligned with said second portion and adapted to contact the exterior of said flexor tendon pulley when extending outwardly from said second section portion.

16. The implant according to claim 15, wherein said at least one leg extends outwardly from said first portion, said first portion including first and second lateral ends and a midpoint between said first and second lateral ends, and said at least one leg includes a first leg, a second leg, and a third leg, said first leg extending outwardly from said first portion at said first lateral end, said second leg extending outwardly from said first portion at said second lateral end, and said third leg extending outwardly from said first portion at said midpoint, said first, second, and third legs adapted to contact and support the interior of the flexor tendon pulley to prevent collapse thereof.

17. The implant according to claim 15, wherein said at least one leg extends outwardly from said second portion, said second portion including first and second lateral ends and a midpoint between said first and second lateral ends, and said at least one leg includes a first leg, a second leg, and a third leg, said first leg extending outwardly from said second portion at said first lateral end, said second leg extending outwardly from said second portion at said second lateral end, and said third leg extending outwardly from said second portion at said midpoint, said first, second, and third legs adapted to contact the exterior of the flexor tendon pulley.

18. An implant for implantation into a human hand for protecting at least one end of a severed flexor tendon from contacting an edge of a flexor tendon pulley, the implant comprising:
a lip portion formed from a material suitable for implantation into the human hand and adapted for placement in contact with the edge of the flexor tendon pulley, said lip portion having an at least in part arcuate shape and a cross section transverse to the at least in part arcuate shape, said lip portion having a channel with an at least in part U-shape along the cross section of said lip portion, said channel defined by a first portion, a second portion, and a connecting portion extending between said first portion and said second portion, said first portion and said second portion having lengths no greater than 3 mm along the cross section of said lip portion, said channel configured to receive the edge of the flexor tendon pulley, wherein, when the edge is received in said channel, said first portion contacts the interior of the flexor tendon pulley and said second portion contacts the exterior of the flexor tendon pulley.

19. The implant according to claim 18, further comprising at least one leg extending outwardly from said first portion of said lip portion, said at least one leg being at least in part aligned with said first portion, said at least one leg adapted to contact the interior of said flexor tendon pulley.

20. The implant according to claim 19, wherein said first portion includes first and second lateral ends and a midpoint between said first and second lateral ends, and said at least one leg includes a first leg, a second leg, and a third leg, said first leg extending outwardly from said first portion at said first lateral end, said second leg extending outwardly from said first portion at said second lateral end, and said third leg extending outwardly from said first portion at said midpoint, said first, second, and third legs adapted to contact and support the interior of the flexor tendon pulley to prevent collapse thereof.

21. The implant according to claim 18, further comprising at least one leg extending outwardly from said second portion of said lip portion, said at least one leg being at least in part aligned with said second portion, said at least one leg adapted to contact the exterior of said flexor tendon pulley.

22. The implant according to claim 21, wherein said second portion includes first and second lateral ends and a midpoint between said first and second lateral ends, and said at least one leg includes a first leg, a second leg, and a third leg, said first leg extending outwardly from said second portion at said first lateral end, said second leg extending outwardly from said second portion at said second lateral end, and said third leg extending outwardly from said second portion at said midpoint, said first, second, and third legs adapted to contact the exterior of the flexor tendon pulley.

23. The implant according to claim 18, further comprising a body portion adapted to be inserted into a passage of the flexor tendon pulley, said body portion having a proximal end, a distal end opposite said proximal end, and a shape adapted to approximate the interior of the passage, said proximal end being at least in part arcuate, said proximal end extending from said first portion of said lip portion.

24. The implant according to claim 23, wherein said body portion is shaped as a partial funnel having an inner portion and an outer portion, said outer portion adapted to support the interior of the flexor tendon pulley to prevent collapse thereof.

25. The implant according to claim 23, wherein said body portion is shaped as a partial cone having an inner portion and an outer portion, said outer portion adapted to support the interior of the flexor tendon pulley to prevent collapse thereof.

26. The implant according to claim 23, wherein said body portion includes at feast one fenestration provided therethrough.
